(19) United States
(12) Patent Application Publication
(10) Pub. No.: US 2011/0163255 A1
(43) Pub. Date: Jul. 7, 2011

(54) METHOD FOR PROTEIN PURIFICATION UNDER DENATURING CONDITIONS

(75) Inventors: Thomas Deutschmann, Wuppertal (DE); Frank Schaefer, Duesseldorf (DE); Helena Block, Hilden (DE)

(73) Assignee: QIAGEN GMBH, 40724 Hilden (DE)

(21) Appl. No.: 13/000,259

(22) PCT Filed: Jun. 26, 2009
(86) PCT No.: PCT/EP2009/004632

§ 371 Date: Mar. 22, 2011

(30) Foreign Application Priority Data
Jun. 27, 2008 (DE) 10 2008 030 142.6

(51) Int. Cl. C09K 15/22; H01F 1/00; C09K 15/32

(52) U.S. Cl. 252/62.51 R; 252/403; 252/400.23

(57) ABSTRACT

The invention relates to a method for the preparation of an application buffer for the purification of proteins by means of immobilized metal ion affinity chromatography (IMAC) under denaturing conditions, which is characterized in that a defined amount of a buffer concentrate having a defined pH value is mixed with a defined amount of a urea concentrate, whereby an application buffer having a defined pH value is provided. According to the invention, a corresponding kit is provided in addition. The components are stable in storage and by mixing produce an application buffer having a defined composition and a defined pH value. The need for pH adjustment or a new preparation is eliminated. The invention can thus be used in an automated manner and as a closed kit concept.

The present invention relates to a method for protein purification and suitable kits and buffer systems.

For the purification of proteins by means of immobilized metal affinity chromatography (subsequently referred to as: IMAC) under denaturing conditions usually guadinium-hydrochloride-containing buffers (see Hochuli et al. 1988) or highly concentrated urea-containing buffer solutions are used. Urea-containing buffer solutions have the advantage over guadinium-hydrochloride-containing buffers that the proteins purified with urea-containing buffers can be directly analyzed on SDS-gels. The optimal binding of the proteins to the matrix, the stringency of the washing steps as well as the efficient elution of the protein of interest critically depend on the pH value of the individual buffer solutions used.

The urea-containing buffer solutions known in the prior art however lose their buffer capacity over time, in particular due to a rise of their pH value. Thereby the stringency of the washing steps and thus the selectivity of the protein purification are reduced. Due to the increased pH value proteins are non-specifically bound to the matrix and can no longer be efficiently washed away. The elution efficiency also decreases. For the purification of recombinant His-tagged proteins via IMAC under denaturing conditions the urea-containing buffer solutions have to be freshly prepared prior to each use or their pH value has to be corrected, i.e. readjusted. This is time consuming and correspondingly disadvantageous.

It is thus the object of the present invention to provide an improved method for protein purification.

The solution to the above problem is provided by the methods, kits and buffer systems specified in claims 1 to 16.

According to one embodiment of the invention, a method for preparing an application buffer for the purification of proteins by means of IMAC under denaturing conditions is provided, which is characterized in that a defined amount of a buffer concentrate having a defined pH value is mixed with a defined amount of a denaturing concentrate, preferably a urea concentrate, whereby an application buffer having a defined pH value is provided.

In the context of the present invention the terms “buffer concentrate” and “application buffer” are used. The term “buffer concentrate” relates to buffer solutions, which are not directly used for purification, but are first mixed with urea to yield the actual application buffer. Preferably, the buffer concentrates do not contain any urea. Initially, urea is present in non-interfering amounts.

The term “application buffer” is used for buffer solutions which are already mixed with urea and can be directly used for protein purification, for example as binding, wash or elution buffer.

The essence of the present invention is the separate provision of the components necessary for protein purification, which react with each other and thereby can result in a pH value increase. According to the invention at least one buffer concentrate having a defined and appropriately preadjusted pH value is provided separately from the urea concentrate. A defined amount of the urea concentrate is mixed just prior to use with a defined amount of puffer concentrate, providing an application buffer having a defined composition and a defined and desired pH value. The advantage of providing separate concentrates is that the single components (buffer concentrate and urea concentrate) are stable during storage and that the pH value of the separately stored components is stable over long periods of time (see FIG. 3). By mixing only two components, namely the buffer concentrate and the urea concentrate, it is possible to obtain an application buffer having a defined composition and a defined pH value even after an extended period of storage of the concentrates (see FIG. 4). In contrast to the prior art the method according to the invention does therefore not require a readjustment of the pH value before or during protein purification. A completely new preparation of the application buffers is also to be dispensed with. The application buffer can be directly used upon mixing of the concentrates. The use of according “ready-to-use” reagents particularly in a kit format reduces work load and saves time for the user. Since according to the invention always an application buffer having the same composition and a defined (identical) pH value is produced, the purification results are reproducible and of constant quality. pH value-induced variations in the application buffers and associated sources of errors are avoided. The method according to the invention is not only user friendly when carried out manually, but also suited for automation, as only the components have to be mixed but no fine tuning or remaking of the application buffers is required. Automation was only poorly realizable with the buffer systems and methods known in the prior art. Furthermore, due to the lack of pH value stability no closed kit concepts suitable for storage were possible (in which no adjustments to the buffer system had to be carried out by the user), since the user had to carry out adjustments to the provided buffers (e.g. adjusting the pH value).

The method according to the invention is in particular used in the purification of His-tagged proteins by means of Ni²⁺-NTA (nickel-nitrilo triacetic acid)-chromatography.

According to one embodiment of the method according to the invention three buffer concentrates are provided, each having a different and already preadjusted pH value. The composition of the buffer concentrates can be identical or different. By mixing these concentrates with a defined amount of urea, at least three application buffers each having a different pH value are obtained. The three basic steps of the method, binding to the matrix, washing the matrix and the subsequent elution of the protein of interest from the matrix each require a buffer having specially adapted, i.e. adjusted pH values (see above). By providing at least three buffer concentrates of different pH values at least three application buffers are provided by mixing with a defined amount of urea concentrate, which not only have a defined composition but also have defined pH values appropriate for the corresponding step. Said application buffers can be directly used, i.e. without further modification, by the user or within an automated method.

According to one embodiment of the method according to the invention, the buffer concentrate used for producing the application buffer for binding has an alkaline pH value, preferably a pH value of from 7.0 to 9.0, particularly preferably a pH value of about 7.5. By mixing of said buffer concentrate with a defined amount of urea concentrate, an application buffer having the same or a higher pH value is produced. According to one embodiment the application buffer for binding has an alkaline pH value, preferably a pH value of from 7.0 to 9.0, particularly preferably a pH value of about 8. The binding buffer can also be used as a lysis buffer and vice versa.

According to this embodiment, the buffer concentrate used for producing the application buffer for washing of the matrix has a slightly acidic pH value, preferably a pH value of from 5.0 to 7.0, particularly preferably a pH value of about 5.6. By mixing this buffer concentrate with a defined amount of urea concentrate, an application buffer having a higher pH value is produced. According to one embodiment the application buffer for washing of the matrix and for releasing non-specifically bound proteins has a slightly acidic to neutral pH value, preferably a pH value of from 5.5 to 7, particularly preferably a pH value of from 6 to 6.5.

The buffer concentrate used for providing the application buffer for elution of the protein according to one embodiment has an acidic pH value, preferably a pH value below 4, particularly preferably a pH value of below 3.5. By mixing of said buffer concentrate with a defined amount of urea concentrate, an application buffer having a higher pH value is produced. According to one embodiment the application buffer for elution of the protein to be purified from the matrix has an acidic pH value, preferably a pH value of below 5.5, particularly preferably a pH value of below 5, e.g. of about 4.5.

The pH values of said buffer concentrates are stable over long periods of time, as can be seen from FIG. 3. One important reason for this pH-stability in comparison to the buffer solutions known in the prior art is the absence of interfering amounts of urea in the buffer concentrates. The application buffers obtain, by purposefully mixing certain amounts of buffer concentrate and urea concentrate, a pH value that may be predetermined or that is defined. Due to this pH-stability and uniformity of the composition the results of the protein purification method become reproducible and comparable in terms of quality. As the user does not have to make up the buffers himself, variations in the composition are avoided. The pH values of the application buffers produced from the concentrates are constant even after extended storage of the concentrates, as can bee seen from FIG. 4.

The buffer concentrates preferably feature a biological buffer. Biological buffers are used in diverse forms in the area of biology and molecular biology. In contrast to classical, inorganic buffering substances they do not have a negative impact on the system to be examined. Biological buffers often contain zwitter ions. An overview of biological buffers can for example be found on the homepage of Sigma-Aldrich (www.sigmaaldrich.com).

According to one embodiment the buffer concentrate for the preparation of the binding application buffer features a buffer having a buffer capacity in the alkaline range, preferably in a range of about 7.0 to 9.0. HEPES (2-(4-(2-hydroxyethyl)-1-piperazinyl)-ethane sulfonic acid), MES (2-(N-morpholino)ethane sulfonic acid), sodium phosphate, citrate, succinate or acetate buffers are to be mentioned as possible buffers, which are usable according to the invention. Amine-containing buffers are preferably used. A preferred example for a biological, amine-containing buffer is a Tris buffer.

According to one embodiment the buffer concentrates for the preparation of the washing and elution application buffer feature a buffer having a buffering capacity in the neutral to acidic range, preferably in a range of about 4.0 to 7.5. Citrate, succinate or acetate buffers are suitable according to the invention. An especially suited example for a biological buffer is Bis-Tris.

The buffer concentrates for preparing the washing buffer and the elution buffer may have the same composition, wherein said buffers however do have a different pH value. The pH value of the wash concentrate is higher than that of the elution concentrate.

According to one embodiment the buffer concentrates in addition feature salts, for example, NaCl, KCl or KH₂PO₄, preferably NaH₂PO₄ and/or non-ionic surfactants, for example Triton X-100, Triton X-114, NP-40, CHAPS, DDM, b-OG, NG, Brij35 or digitonin or preferably Tween. According to one embodiment the buffers additionally contain a preservative, preferably NaN₃.

According to one embodiment of the method according to the invention the buffer concentrate used for the preparation of the application buffer contains 25-500 mM, preferably 50-300 mM, particularly preferred 100-250 mM Tris(hydroxymethyl)-aminomethane (in particular for preparing the binding buffer) or BisTris(Bis(2-hydroxyethyl)amino-tris(hydroxymethyl)methane (in particular for preparing the washing or elution buffer), as well as additionally 125-750 mM, preferably 250 to 500 mM, particularly preferred 300-450 mM NaH₂PO₄ and/or 0.025 to 0.5% (v/v) preferably 0.05-0.3% (v/v), particularly preferred 0.1 to 0.25% (v/v) non-ionic surfactants and/or 0.0025 to 0.05%, preferably 0.005-0.03%, particularly preferred 0.01 to 0.025% NaN₃. Preferably used are Tris(hydroxymethyl)-aminomethane or BisTris(Bis(2-hydroxyethyl)amino-tris(hydroxymethyl)methane in the form of Tris(hydroxymethyl)-aminomethane chloride or BisTris(Bis(2-hydroxyethyl)amino-tris(hydroxymethyl)methane chloride.

According to one embodiment of the method according to the invention the urea concentrate separately provided for the preparation of the application buffer is an aqueous solution of 8 to 10 M urea; according to a preferred embodiment the urea concentrate is an aqueous solution of 9.3 to 9.8 M urea, preferably 9.6 M urea.

According to one embodiment one part of the buffer concentrate is mixed with 3.7 parts of the urea concentrate to obtain an application buffer according to the invention. According to one embodiment this mixing ratio is used for the preparation of all application buffers.

The invention further relates to the use of a urea concentrate for the preparation of an application buffer having a defined pH value, wherein a defined amount of a urea concentrate is mixed with a defined amount of a buffer concentrate. By separately providing the urea concentrate and its mixing with the buffer concentrate just prior to use, application buffers are obtained that not only have a defined composition but also have a defined, pre-determined pH value. This contributes to improved conditions for protein purification which above all may be standardized. Details of the advantages, the composition of the urea and buffer concentrates have been discussed in detail above. We refer to the above disclosure, which applies also with respect to the use.

According to a particular embodiment a kit for the purification of proteins by IMAC under denaturing conditions is provided, which has at least a buffer concentrate having a defined pH value and a urea concentrate for the preparation of at least one application buffer having a defined pH value.

According to one embodiment the kit has at least three buffer concentrates each having a different and defined pH value, which by mixing with a defined amount of urea concentrate respectively result in at least three different application buffers having a defined composition and a defined pH value.

The kit according to the invention is stable due to the separately provided concentrates and thus advantageously suited for storage. Through the small number of components that can be directly used the kit is well-arranged and is thus safely and quickly usable for the user. It is not necessary for the user to adjust the buffer conditions, whereby reproducible and qualitatively constant results are achieved. The kit can also be used by personnel less well trained as it is less error prone. It is thus especially suited for a high throughput of samples. Additionally, the kit can be used in combination with an automated method.

The kit can also be a buffer system for the purification of proteins by IMAC under denaturing conditions, which has at least a buffer concentrate having a defined pH value and a urea concentrate, which by mixing with the buffer concentrate yields an application buffer having a defined pH value.

Details with respect to the buffer concentrates, the urea concentrate as well as to the application buffers prepared therefrom are provided above. We refer to the above statements, which also apply in the context of the kit and are part of the respective disclosure.

According to one embodiment of the kit according to the invention the buffer concentrate used for the preparation of the application buffers contains 25-500 mM, preferably 50-300 mM, especially preferred 100-250 mM Tris(hydroxymethyl)-aminomethane (in particular for the preparation of the binding buffer) or BisTris(Bis(2-hydroxyethyl)amino-tris(hydroxymethyl)methane (in particular for the preparation of the washing or elution buffer), as well as additionally 125-750 mM, preferably 250 to 500 mM, particularly preferred 300-450 mM NaH₂PO₄ and/or 0.025 to 0.5% (v/v), preferably 0.05-0.3% (v/v), particularly preferred 0.1 to 0.25% (v/v) non-ionic surfactants and/or 0.0025 to 0.05%, preferably 0.005-0.03%, particularly preferred 0.01 to 0.025% NaN₃. Preferably used is Tris(hydroxymethyl)-aminomethane or BisTris(Bis(2-hydroxyethyl)amino-tris(hydroxymethyl)methane in the form of Tris(hydroxymethyl)-aminomethane chloride or BisTris(Bis(2-hydroxyethyl)amino-tris(hydroxymethyl)methane chloride.

According to one embodiment of the kit according to the invention the urea concentrate is an aqueous solution of 8 to 10 M urea; according to a preferred embodiment the urea concentrate is an aqueous solution of 9.3 to 9.8 M urea, preferably 9.6 M urea.

According to one embodiment the kit components (buffer concentrate(s), urea concentrate) change their pH value by no more than +/−0.5, preferably by +/−0.3 or +/−0.1. They are therefore suitable for storage and can be used as a closed kit concept.

The kit components suited for storage preferably yield application buffers of a defined pH value+/−0.5 upon mixing the buffer concentrates with the urea concentrate after a storage period of at least 100, preferably 150 days.

The kit can further feature a MAC matrix, preferably a Ni²⁺-NTA matrix. Said matrix can also be provided in form of beads, in particular magnetic beads.

### FIGURES

FIG. 1: The problems of the lacking pH-stability of buffer systems known in the prior art, which are used as a standard for the denaturing purification of 6×His-tagged proteins are demonstrated. The following buffer compositions were used for the binding, wash and elution buffer (application buffer):

100 mM NaH₂PO₄, 10 mM Tris, 8 M urea and 0.05% Tween 20. The pH value of the binding buffer (buffer B-T) was adjusted to pH 8.0, that of the washing buffer (buffer C-T) to pH 6.3 and that of the elution buffer (buffer E-T) to pH 4.5. As shown in FIG. 1a) to c) the pH value of the individual buffer solutions already increases linearly after a few days.

FIG. 1 a) depicts the pH value change of a urea-containing binding buffer solution “buffer B-T” for binding of the protein to the matrix. As clearly evidenced in the graph, the pH value of the buffer solution which was adjusted to 8.0 starts to increase linearly after a few days and reaches a pH value of 8.4 after about two months.

FIG. 1 b) displays the changes of the pH value for a urea-containing application buffer “buffer C-T” for washing and removing non-specifically bound proteins from the matrix. As clearly evident from the graph the pH value, which was initially adjusted to 6.3, already rises linearly after a few days and reaches a neutral pH value after about 2 months already. Washing the matrix is no longer efficient under these conditions; the pH value has to be re-adjusted.

FIG. 1 c) depicts the pH value change in a urea-containing application buffer “buffer E-T” for elution of the proteins of interest from the matrix. As clearly evident from the graph the pH value, which was initially adjusted to 4.5, already rises linearly after a few days and reaches a pH value above 6.5 after about two months already. In this case an efficient elution of the protein is no longer possible, the pH value has to be re-adjusted.

Due to the pH rise binding of the protein to the matrix is no longer optimal; furthermore the stringency of the washing steps is impaired and the elution efficiency is reduced. The use of these buffers over an extended period of time would require regular pH re-adjustments.

FIG. 2: The rise of the pH values in the urea-containing buffer solutions could be due to the depicted order of reactions. Urea could form a reaction intermediate with Tris, from which ammonia, a strong base, is then split off. Due to the developing ammonia the pH value of the buffer solution is quickly shifted to the basic range. In addition, through the reaction of the urea with the Tris, Tris molecules are lost, which can then no longer contribute to the buffer capacity.

FIG. 3: The pH value stability of the buffer concentrates according to the invention is demonstrated. Eight different pH measurements were performed at different time points. The following buffer compositions were chosen for the buffer concentrates: 370 mM NaH₂PO₄, 185 mM TrisCl, 0.185% (v/v) Tween20, 0.02% NaN₃ for the binding buffer concentrate; 370 mM NaH₂PO₄, 185 mM Bis-TrisCl, 0.185% (v/v) Tween20, 0.02% NaN₃ for the washing and the binding buffer concentrate.

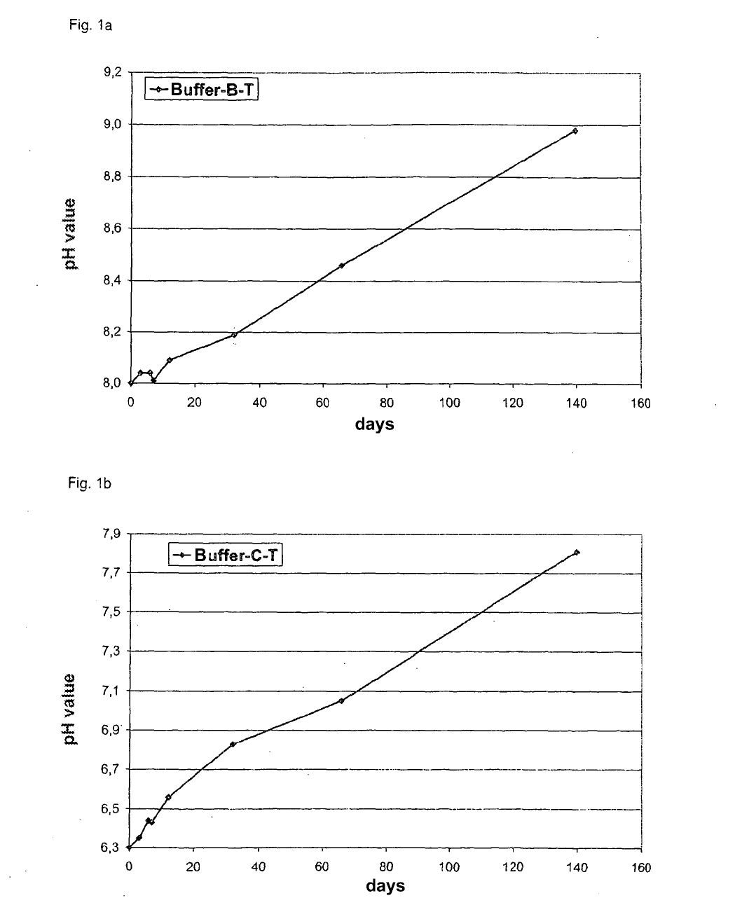

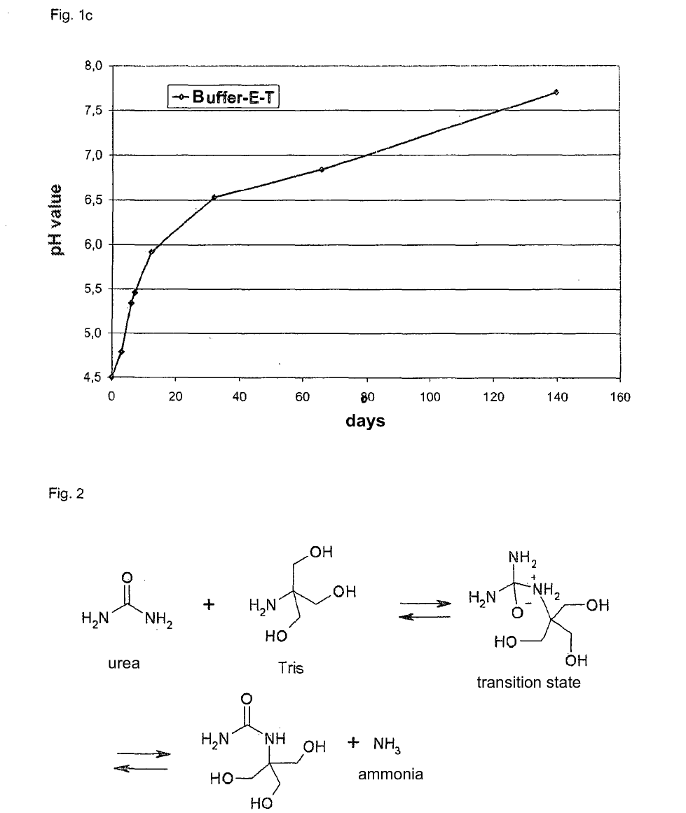

The binding buffer concentrate preadjusted to pH 7.5 (here: NTT-7.5) remained at its pH value without fluctuations even after more than 100 days. The wash buffer concentrate preadjusted to pH 5.6 (here NTT-5.6) also remained at its pH value without fluctuations or appreciable changes of the pH value even after more than 100 days. The binding buffer concentrate preadjusted to pH 3.0 (here: NTT-3.0) also remained at its pH value even after more than 100 days without appreciable fluctuations. This experiment demonstrates the pH stability of the concentrates used according to the invention, whereby they may be used within closed kit systems due to their storage stability.

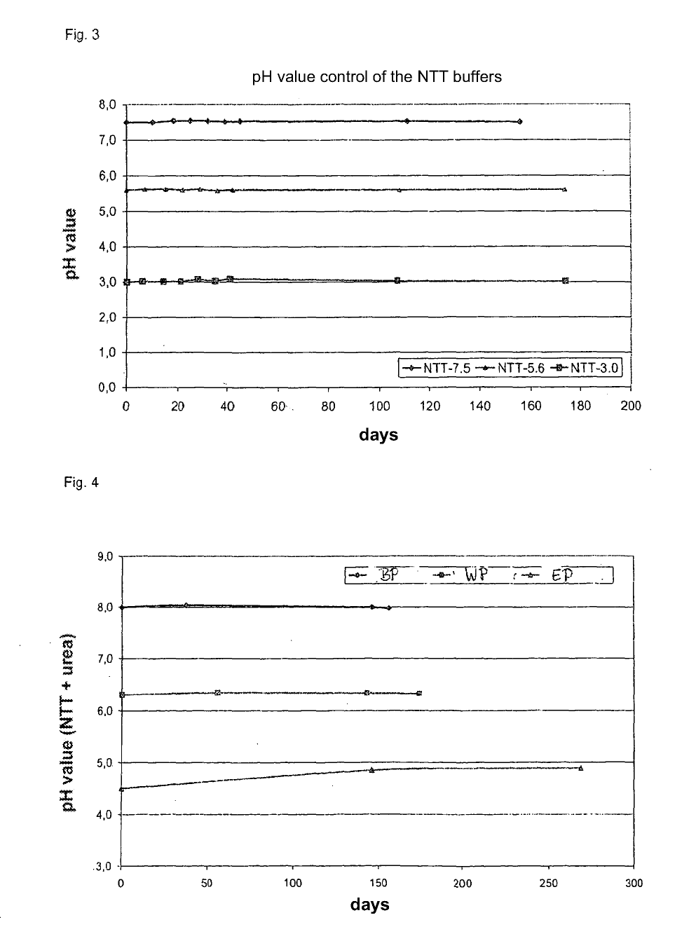

FIG. 4: An example for the pH stability of the application buffers (BP=binding buffer; WP=washing buffer; EP=elution buffer) prepared according to the invention from the concentrates (buffer concentrate and urea concentrate) is depicted. For the preparation of the application buffers, the buffer concentrates with the compositions and pH values described in FIG. 3 were chosen. The urea concentrate consisted of an aqueous solution of 9.6 M urea, such that the final application buffers contained 7.0 M urea.

The application buffer for binding (pH 8.0) is made by mixing a defined amount of binding buffer concentrate of pH 7.5 (see above) with the urea concentrate. The application buffer for washing (pH 6.3) is made by mixing the wash buffer concentrate (see above) with the urea concentrate. Each time the application buffers were mixed prior to the measurement from the stored concentrates (buffer concentrate and urea concentrate). The application buffers for binding and for washing made from the concentrates did not show any appreciable pH value changes up to day 150. The same applies to the elution buffer.

The application buffer for elution (pH 4.5) is made by mixing a defined amount of buffer concentrate having pH 3.0 (see above) with the urea concentrate. Over a period of measurement of more than 250 days the elution buffer concentrate shows only a minor, tolerable pH value increase from 4.5 to about 4.9. This demonstrates that even after extended storage, application buffers having a defined composition and a defined, desired pH value are obtained from the individual concentrates by simple mixing. The advantages were described in detail above.

1. Method for preparing an application buffer for the purification of proteins by immobilized metal affinity chromatography (IMAC) under denaturing conditions, said method comprising mixing a defined amount of a buffer concentrate having a defined pH value with a defined amount of a urea concentrate, whereby an application buffer having a defined pH value is provided.

2. Method according to claim 1, wherein at least three buffer concentrates each having a different pH value are mixed with a defined amount of urea concentrate, whereby three different application buffers are provided, each having a different, defined pH value.

3. Method according to claim 1, comprising one or more of the following features:
a. that the buffer concentrate, which after mixing with the urea concentrate is an application buffer for binding, has an alkaline pH value; and/or
b. that the buffer concentrate, which after mixing with the urea concentrate is an application buffer for binding, has a pH value of from 7.0 to 9.0; and/or
c. that the buffer concentrate, which after mixing with the urea concentrate is an application buffer for binding, has a pH value of about 7.5; and/or
d. that the buffer concentrate, which after mixing with the urea concentrate is an application buffer for washing, has a slightly acidic pH value; and/or
e. that the buffer concentrate, which after mixing with the urea concentrate is an application buffer for washing, has a pH value of from 5.5 to 7.0; and/or
f. that the buffer concentrate, which after mixing with the urea concentrate is an application buffer for washing, has a pH value of about 5.6; and/or
g. that the buffer concentrate, which after mixing with the urea concentrate is an application buffer for elution, has an acidic pH value; and/or
h. that the buffer concentrate, which after mixing with the urea concentrate is an application buffer for elution, has a pH value of not more than 4.

4. Method according to claim 1, comprising one or more of the following features
a. that the application buffer for binding produced by mixing the buffer concentrate with the urea concentrate has an alkaline pH value; and/or
b. that the application buffer for binding produced by mixing the buffer concentrate with the urea concentrate has a pH value of from 7.0 to 9.0; and/or
c. that the application buffer for binding produced by mixing the buffer concentrate with the urea concentrate has a pH value of about 8; and/or
d. that the application buffer for washing produced by mixing the buffer concentrate with the urea concentrate has a slightly acidic to neutral pH value; and/or
e. that the application buffer for washing produced by mixing the buffer concentrate with the urea concentrate has a pH value of from 5.5 to 9.0; and/or
f. that the application buffer for washing produced by mixing the buffer concentrate with the urea concentrate has a pH value of from 6 to 6.5; and/or
g. that the application buffer for elution produced by mixing the buffer concentrate with the urea concentrate has an acidic pH value; and/or
h. that the application buffer for elution produced by mixing the buffer concentrate with the urea concentrate has a pH value of not more than 5.5.

5. Method according to claim 1, wherein the buffer concentrate having a defined pH value features a biological buffer.

6. Method according to claim 1, wherein
a. the buffer concentrate for the preparation of the binding application buffer features a buffer having a buffer capacity in the alkaline range; and/or
b. the buffer concentrate for the preparation of the washing and application buffer features a buffer having a buffer capacity in the neutral to acidic range; and/or
c. the buffer concentrate features a salt.

7. Method according to claim 1, wherein as a urea concentrate an aqueous solution containing 8 to 10 M, urea is used.

8. Method according to claim 1, wherein at least one buffer concentrate and the urea concentrate are mixed in a ratio of 1 to 3.7.

9. A urea concentrate suitable for preparation of an application buffer having a defined pH value, wherein a defined amount of said urea concentrate has been mixed with a defined amount of a buffer concentrate having a preadjusted pH value.

10. Kit for purifying proteins by means of immobilized metal affinity chromatography (IMAC) under denaturing conditions comprising at least one buffer concentrate having a defined pH value and at least a urea concentrate for preparing an application buffer having a defined pH value.

11. Kit according to claim 10 comprising at least three buffer concentrates having a different, defined pH value and a urea concentrate for the preparation of at least three application buffers each having a different, defined pH value.

12. Kit according to claim 10, comprising one or more of the following features:
a. the buffer concentrate, which after mixing with the urea concentrate is an application buffer for binding, has an alkaline pH value; and/or
b. the buffer concentrate, which after mixing with the urea concentrate is an application buffer for binding, has a pH value of from 7.0 to 9.0; and/or
c. the buffer concentrate, which after mixing with the urea concentrate is an application buffer for binding, has a pH value of 7.5; and/or
d. the buffer concentrate, which after mixing with the urea concentrate is an application buffer for washing, has a slightly acidic to neutral pH value; and/or
e. the buffer concentrate, which after mixing with the urea concentrate is an application buffer for washing, has a pH value of from 5.5 to 7; and/or
f. the buffer concentrate, which after mixing with the urea concentrate is an application buffer for washing, has a pH value of about 5.6; and/or
g. the buffer concentrate, which after mixing with the urea concentrate is an application buffer for elution, has an acidic pH value; and/or
h. the buffer concentrate, which after mixing with the urea concentrate is an application buffer for elution, has a pH value of not more than 4.

13. Kit according to claim 10, characterized in that the buffer concentrate having a defined pH value features a biological buffer.

14. Kit according to claim 10, wherein
a. the buffer concentrate for the preparation of the binding application buffer features a buffer having a buffer capacity in the alkaline range; and/or
b. the buffer concentrate for the preparation of the washing and application buffer feature a buffer having a buffer capacity in the neutral to acidic range; and/or
c. the buffer concentrate features a salt.

15. Kit according to claim 10, comprising a buffer system.

16. Kit according to claim 10, comprising one or more of the following features:
a. the kit components change their pH value for at least 100 days by no more than +/−0.1; and/or
b. the kit components yield application buffers of a defined pH value+/−0.5 upon mixing the buffer concentrates with the urea concentrate after a storage period of at least 100 days; and/or
c. the kit features a matrix NTA; and/or
d. the kit is suitable for automated use.

17. Kit according to claim 10, wherein the kit comprises an IMAC matrix optionally in the form of magnetic particles.

18. A method of claim 6, wherein the buffer having buffer capacity in the alkaline range comprises Tris and the buffer having a buffer capacity in the neutral to acidic range comprises Bis-Tris and the buffer concentrate featuring a salt comprises NaH₂PO₄ and/or detergents, and/or a preservative.

19. A kit of claim 14, wherein the buffer having buffer capacity in the alkaline range comprises Tris and the buffer having a buffer capacity in the neutral to acidic range comprises Bis-Tris and the buffer concentrate featuring a salt comprises NaH₂PO₄ and/or detergents, and/or a preservative.
